(19) United States
(12) Patent Application Publication
(10) Pub. No.: US 2026/0248516 A1
(43) Pub. Date: Aug. 27, 2026

(54) Total Ankle Replacement Alignment Reference Wing and Sizing Guides

(71) Applicant: Arthrex, Inc., Naples, FL (US)

(72) Inventors: Maris Prieditis, Naples, FL (US); Adam N. Garlock, Naples, FL (US); Benjamin Chan, Bonita Springs, FL (US); Braham Dhillon, Naples, FL (US); Brandton Ferrin, Bonita Springs, FL (US)

(21) Appl. No.: 18/727,371

(22) PCT Filed: Jan. 23, 2023
(86) PCT No.: PCT/US2023/061073

§ 371 Date: Jul. 9, 2024

Related U.S. Application Data
(60) Provisional application No. 63/301,840, filed on Jan. 21, 2022.

(51) Int. Cl. A61B 17/15; A61B 17/17; A61F 2/42

(52) U.S. Cl. CPC A61B 17/15; A61B 17/1775; A61F 2/4202; A61F 2002/4205; A61F 2002/4207

(57) ABSTRACT

A device comprising: a reference wing having a first end and a second end opposite the first end, wherein each of the first end and the second end of the reference wing includes a plurality of through holes; a resection profile configured to be removably coupled to the reference wing between the first end and the second end; and a lateral rod configured to be removably coupled to the reference wing via the plurality of through holes.

### CROSS-REFERENCE TO RELATED APPLICATIONS

This application claims the benefit of priority to U.S. Provisional Application No. 63/301,840 entitled “Total Ankle Replacement Alignment Reference Wing and Sizing Guides,” filed on Jan. 21, 2022, the contents of which are hereby incorporated by reference in its entirety.

### BACKGROUND

In total ankle replacement (TAR) arthroplasty, surgeons size and position the tibial and talar implants in the anterior-posterior (AP) and lateral views relative to the both the native bone anatomy and the joint line. In addition, surgeons need to be able to do this while ensuring they are looking at the true view of the bone anatomy. The present disclosure provides a system for sizing and positioning ankle implants.

### SUMMARY

The present disclosure includes instruments that aid in sizing and positioning ankle implants.

In particular, the present disclosure includes a system that assists in the aligning of the TAR external adjustment guide and the K-wire placement required to secure the bone resection blocks. Under fluoroscopy, a reference wing and associated attachments indicate the position of the bone resection profile and overall implant placement. When the proper alignment is present, the reference wing and attachments will form thin line profiles that only show their thickness under fluoroscopy. A secondary alignment check is present when a horizontal line of the resection profile and a vertical line of the reference profile intersect to form a crosshair. The reference wing is connected to the TAR external adjustment guide without the need to manually engage mechanical fasteners and allows lateral guides and different sized implant outline guides to be attached to the wing without manually engaging mechanical fasteners. The quick connect feature is designed to be infinitely adjustable, in the anterior-posterior direction, while providing enough force to hold the mating attachments in place during the alignment technique.

Not only does the system described herein allow a surgeon to assess the varus/valgus rotation in the anteriorposterior plane and flexion/extension rotation in the sagittal plane, but it allows the surgeon to determine implant size and position, both relative the respective tibial and talar bones individually, but also as a coupled cut relative to the joint line.

In existing systems, the reference alignment wing is used for both varus/valgus and coronal alignment. However, these existing systems do not provide bone resection lines or implant sizing reference lines to indicate the implant placement as part of the alignment wing. Existing systems also rely on mechanical attachment mechanisms that do not allow for infinite adjustment in the anterior-posterior direction. Traditional alignment wings indicate the superior tibial cut only while the system of the present disclosure allows the surgeon to check the superior tibial cut along with sizing the implant medial-lateral in an anterior view, while also checking resection heights for the tibia and talus resection in the lateral view as well.

Thus, in a first aspect, a device includes a reference wing having a first end and a second end opposite the first end. Each of the first end and the second end of the reference wing includes a plurality of through holes. The device further includes a resection profile configured to be removably coupled to the reference wing between the first end and the second end. The device further includes a lateral rod configured to be removably coupled to the reference wing via the plurality of through holes.

In another aspect, the present disclosure provides a device including a gutter sword including a flat portion configured to be positioned between a tibia and a talus of a patient and an elongated rod extending from the flat portion. The device also includes a link including a first through hole configured to receive the elongated rod of the gutter sword and a plurality of second through holes extending away from the first through hole. The device also includes a pin alignment guide including a rod configured to be positioned in one of the plurality of second through holes of the link. The pin alignment guide includes one or more through holes configured to receive one or more pins therethrough.

In another aspect, a kit can include the device of the first aspect and the device of the second aspect.

In yet another aspect, a method of preparing a tibia for an implant can include positioning a pin in the tibia of a patient, positioning an external adjustment guide over the pin, removably coupling a reference wing to the external adjustment guide, removably coupling a resection profile to the reference wing, adjusting, via the external adjustment guide, a position of the resection profile with respect to the tibia, and resecting a portion of the tibia using the resection profile as a guide.

### BRIEF DESCRIPTION OF THE DRAWINGS

FIG. 1 is a perspective view of an example device.

FIG. 2 is an exploded view of the example device of FIG. 1.

FIG. 3 is a perspective view of the reference wing of the example device of FIG. 1.

FIG. 4 is a top cross-sectional view of the reference wing of the example device of FIG. 1.

FIG. 5 is a perspective view of the resection profile of the example device of FIG. 1.

FIG. 6 is a front view of the resection profile of the example device of FIG. 1.

FIG. 7 is side view of the resection profile of the example device of FIG. 1.

FIG. 8 is a front view of a plastic block of another example resection profile.

FIG. 9 is a perspective view of the plastic block of the resection profile of FIG. 8.

FIG. 10 is a front view of a metal insert of the resection profile of FIG. 8

FIG. 11 is a front view of the assembled resection profile with the metal insert of FIG. 10 positioned in the plastic block of FIG. 8.

FIG. 12 is a side view of the reference wing of the example device of FIG. 1 illustrating a vertical alignment line.

FIG. 13 is a side view of the resection profile of the example device of FIG. 1 illustrating a horizontal reference line.

FIG. 14 is a side view of the resection profile placed onto the reference wing of the example device of FIG. 1 illustrating the vertical alignment line of the reference wing and the horizontal reference line of the resection profile forming a crosshairs.

FIG. 15 is perspective view of a biplane of the lateral rod of the example device of FIG. 1.

FIG. 16 is a perspective view of the biplane of FIG. 15 positioned through the reference wing of the example device of FIG. 1 prior to coupling with the lateral blade.

FIG. 17 is perspective view of the lateral blade of the lateral rod of the example device of FIG. 1.

FIG. 18 is a side cross-sectional view of the lateral blade of FIG. 17.

FIG. 19 illustrates an anterior-posterior view of a block configured to be removably coupled to the lateral rod of the example device of FIG. 1.

FIG. 20 illustrates a sagittal view of the block of FIG. 19.

FIG. 21 illustrates an anterior-posterior view of the block of FIG. 19 coupled to the device of Figure I and positioned adjacent an ankle anatomy.

FIG. 22 illustrates a sagittal view of the block of FIG. 19 coupled to the device of FIG. 1 and positioned adjacent an ankle anatomy.

FIG. 23 illustrates a device for initial pin alignment positioned adjacent an ankle anatomy.

FIG. 24 illustrates another device for initial pin alignment.

FIG. 25 illustrates an alignment system including an external adjustment guide and reference wing positioned adjacent an ankle anatomy.

FIG. 26 illustrates a knee clamp of the alignment system of FIG. 25.

### DETAILED DESCRIPTION

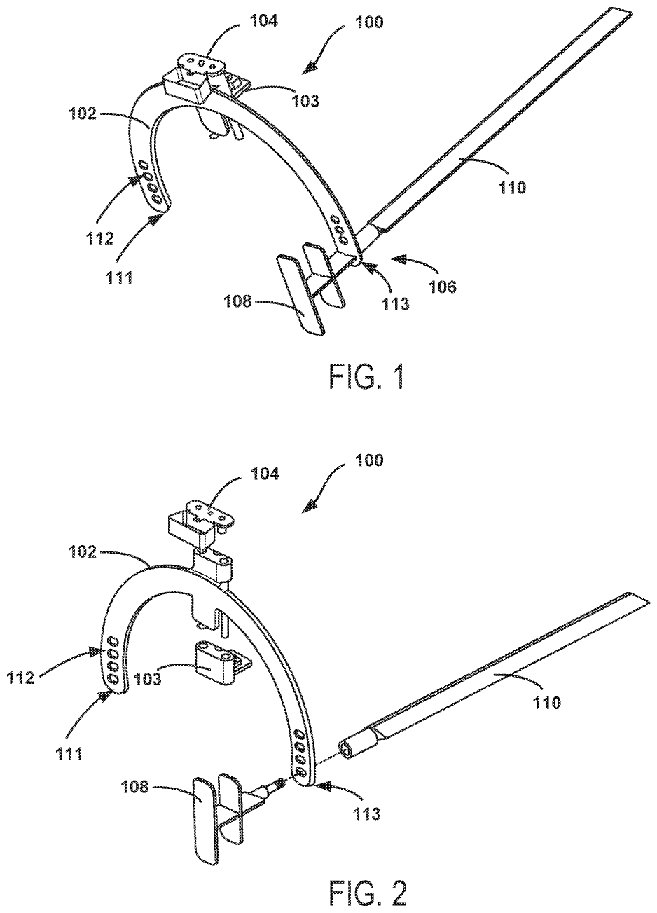

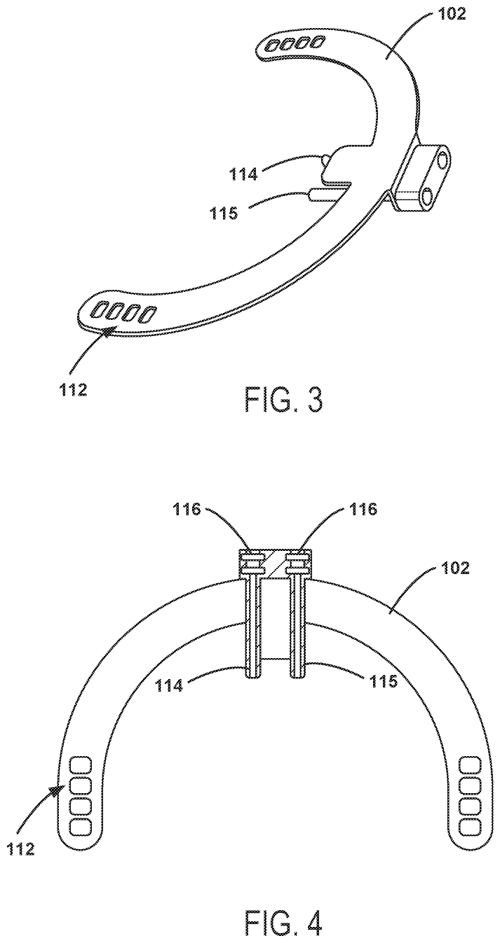

With reference to the Figures, FIGS. 1-2 show a device 100. The device 100 includes a reference wing 102, a resection profile 104, and a lateral rod 106. The lateral rod 106 includes a biplane 108 that is removably coupled a lateral blade 110. As shown in FIGS. 1-2, the reference wing 102 is made of a semicircular blade with a plurality of through holes 112 each of a first end 111 and a second end 113 of the reference wing 102 for mating with the lateral rod 106.

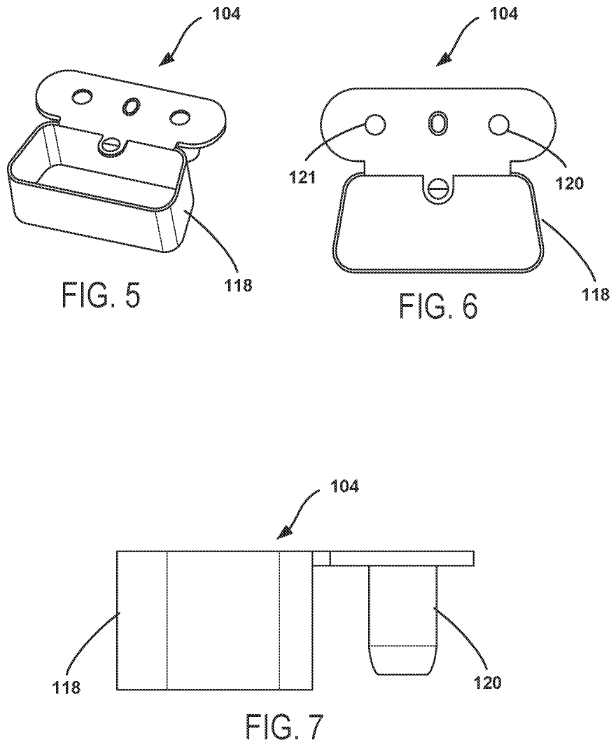

FIG. 4 illustrates the reference wing 102 including a first cannulated rod 114 and a second cannulated rod 115, each positioned between the first end 111 and the second end 113 of the reference wing. The resection profile 104 includes a first rod 120 and a second rod 121 configured to mate within the first cannulated rod 114 and the second cannulated rod 115 of the reference wing 102 to thereby removably couple the resection profile 104 to the reference wing 102. In one example, the first cannulated rod 114 and the second cannulated rod 115 each include a canted coil spring 116. The reference wing 102 is connected to the resection profile 104 without the need to manually engage mechanical fasteners and allows various resection profiles to be attached to the reference wing 102 without manually engaging mechanical fasteners. The quick connect feature is designed to be infinitely adjustable, in the anterior-posterior direction, while providing enough force to hold the resection profile 104 in place during the alignment technique. As shown in FIGS. 5-7, the resection profile 104 is created by a thin wall 118 that outlines the area of the anatomy that will be resected. The resection profile 104 include the first rod 120 and the second rod 121 that mate with the canted coil springs 116 on the reference wing 102.

The device 100 may further include an external adjustment guide 103 configured to be removably coupled to the reference wing 102 between the first end 111 and the second end 113 of the reference wing 102. The external adjustment guide 103 is configured to be positioned on a first side of the reference wing 102, and the resection profile 104 is configured to be positioned on a second side of the reference wing 102, as shown in FIGS. 1-2. As described above, the reference wing 102 may include a first cannulated rod 114 and a second cannulated rod 115 each positioned between the first end 111 and the second end 113 of the reference wing 102. The external adjustment guide 103 may include a first hole 117 and a second hole 119 configured to receive the first cannulated rod 114 and the second cannulated rod 115 of the reference wing 102 to thereby removably couple the external adjustment guide 103 to the reference wing 102. In one example, the first hole 117 and the second hole 119 each include a canted coil spring. As such, the reference wing 102 is connected to the external adjustment guide 103 without the need to manually engage mechanical fasteners and allows lateral guides and different sized implant outline guides to be attached to the reference wing 102 without manually engaging mechanical fasteners. The quick connect feature is designed to be infinitely adjustable, in the anterior-posterior direction, while providing enough force to hold the external adjustment guide 103 in place during the alignment technique.

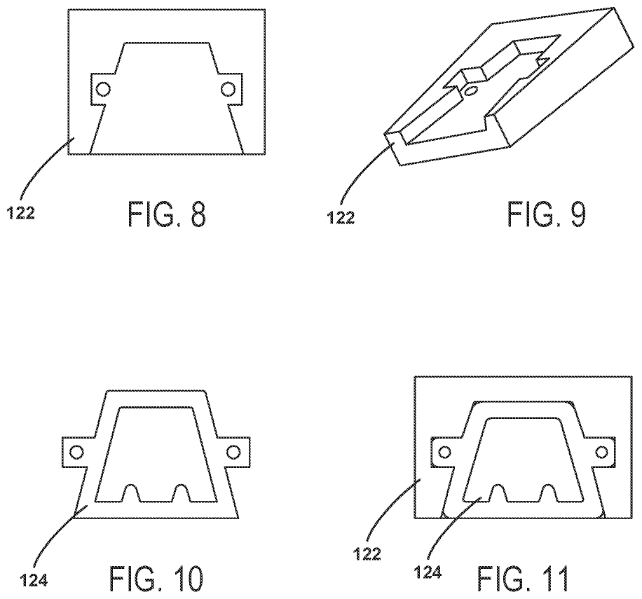

Another variation of the above resection profile 104 is shown in FIGS. 8-11. In particular, FIGS. 8-11 illustrate a two-piece assembly that is attached view threaded fasters. The first piece is a plastic block 122 that has the capability to receive a metal insert 124 that is the shape profile of the TAR bone cut. The metal insert 124 can show the tibial cut, talar cut, and even the joint line in the AP view. The metal insert 124 may or may not include fluoroscopic alignment features. Further, the metal insert 124 may or may not include on-axis and off-axis k-wire hole for pinning the plastic block 122 to the bone. Additional on the plastic block 122 can include an attachment feature (not shown) that allows it to connect to the reference wing 102. The metal insert 124 is not pressed into the plastic block 122. Instead, the metal insert 124 is laid on top of the plastic block 122 and secured with a fastener. By securing the metal insert 124 to the plastic block 122 via a fastener, the residual stress of press fitting pins or through the metal expanding and contacting during sterilization can be avoided.

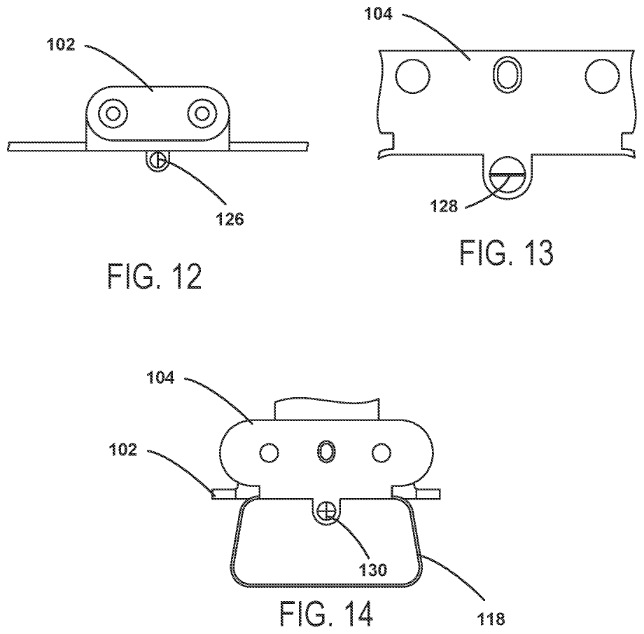

FIG. 12 is a side view of a portion of the reference wing 102 illustrating a vertical alignment line 126. FIG. 13 is a side view of the resection profile 104 illustrating a horizontal alignment line 128. FIG. 14 is a side view of the resection profile 104 placed onto the reference wing 102 illustrating the vertical alignment line 126 of the reference wing 102 and the horizontal alignment line 128 of the resection profile forming a crosshairs 130. When the resection profile 104 is placed onto the reference wing 102 as shown in FIG. 4, the crosshairs 130 enables the user to visualize a true anterior view of the device 100.

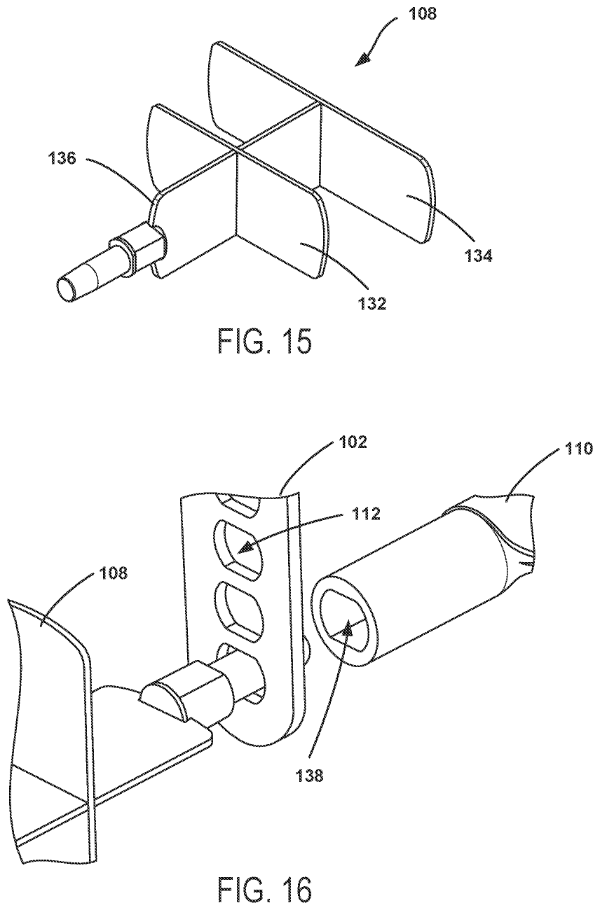

As described above, the lateral rod 106 may be split into two components (biplane 108 and lateral blade 110) that mate together with the reference wing 102. As shown in FIG. 15, the biplane 108 can include two horizontal blades 132, 134 and a vertical blade 136. The two horizontal blades 132, 134 indicate the superior talar resection and native joint line, respectively.

FIG. 16 is a perspective view of the biplane 108 positioned through the reference wing 102 prior to coupling with the lateral blade 110. As shown in FIG. 16, the biplane 108 can include a shape feature that matches the plurality of holes 112 on the reference wing 102 and further matches the mating hole 138 of the lateral blade 110. The shape feature may prevent rotation of the biplane 108 with respect to the reference wing 102 when the shape feature is positioned through one of the plurality of holes 112.

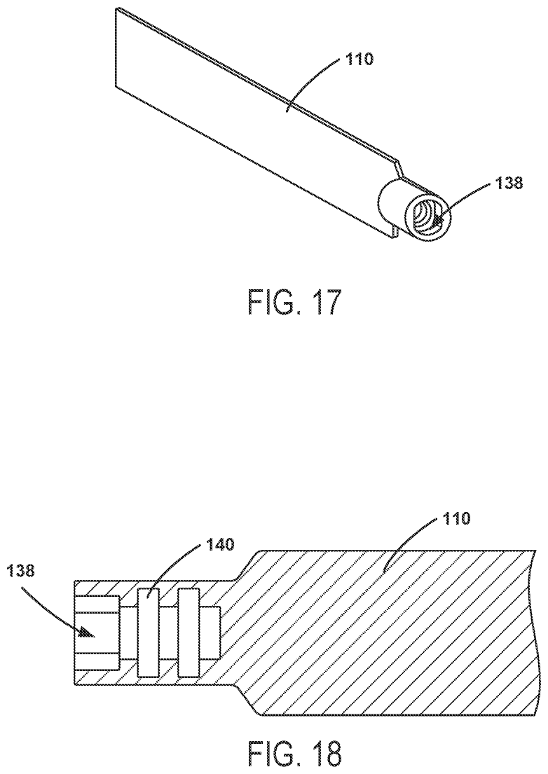

FIG. 17 is perspective view of the lateral blade 110 of the lateral rod 106, and FIG. 18 is a side cross-sectional view of the lateral blade 110. As shown in FIG. 18, the mating hole 138 of the lateral blade 110 may include canted coil springs 140 that will hold the biplane 108 in place on the reference wing 102. The lateral blade 110 has a thin blade profile that aligns with the tibial axis when in use.

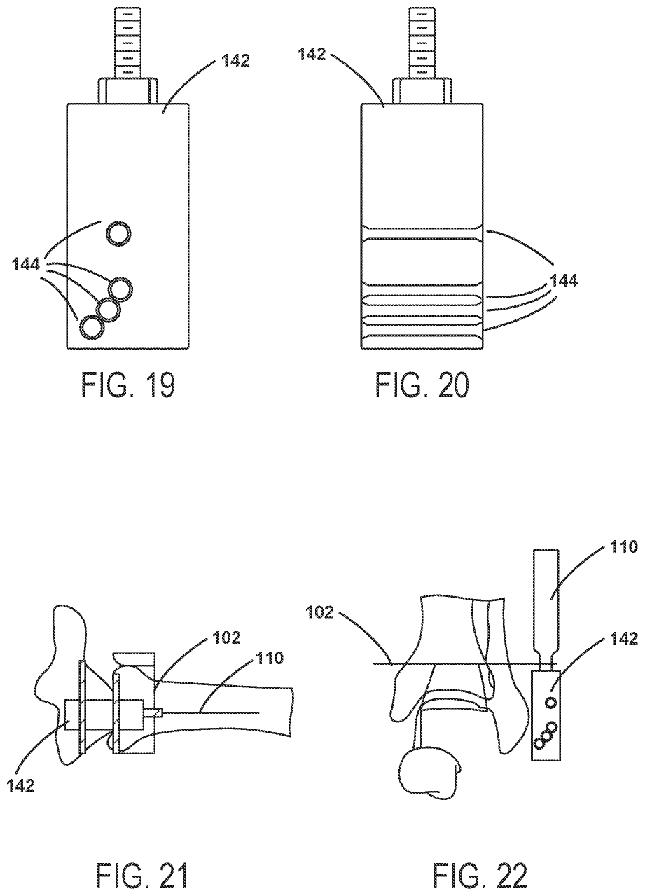

FIGS. 19-22 illustrate another example component that can be removably coupled to the lateral blade 110 in place of the biplane 108. As shown in FIGS. 19-22, a block 142 is shown with k-wire holes 144. The block 142 attaches to the reference wing 102 is a similar fashion as the biplane 108 (e.g., via the canted coil springs 116 of the lateral blade 110). The k-wire holes 144 are placed such that they reference the joint line, and talar cut for multiple sizes of implant. In an example, the tibial cut could be referenced by the block 142. In another example, the tibial cut is shown by the reference wing 102. The block 142 may be made from a transparent material under fluoroscopy (i.e. radel, etc.), and may be used to tell different size talar flat cuts, chamfer cuts, or both.

FIG. 19 illustrates an anterior-posterior view of the block 142. FIG. 20 illustrates a sagittal view of the block 142. FIG. 21 illustrates an anterior-posterior view of the block 142 coupled to the lateral blade 110 and positioned adjacent an ankle anatomy. FIG. 22 illustrates a sagittal view of the block of FIG. 19 coupled to the lateral blade 110 and positioned adjacent an ankle anatomy.

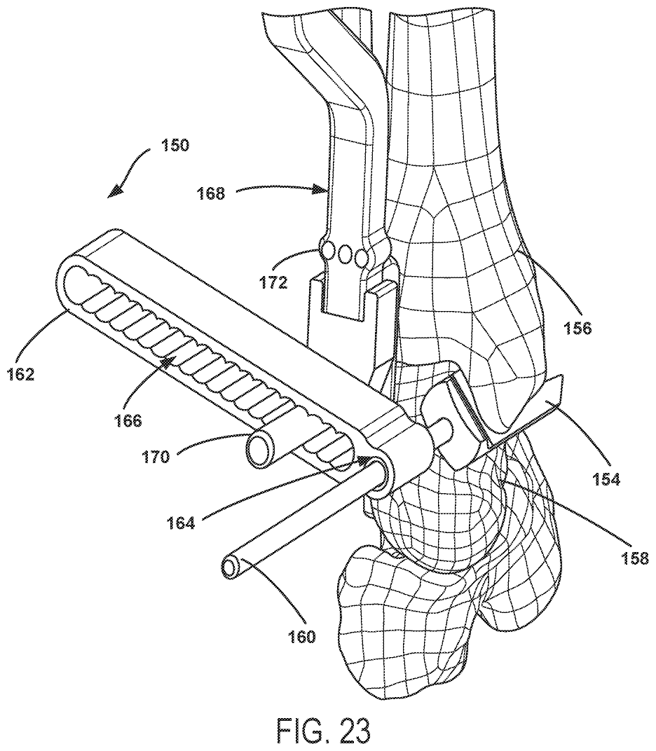

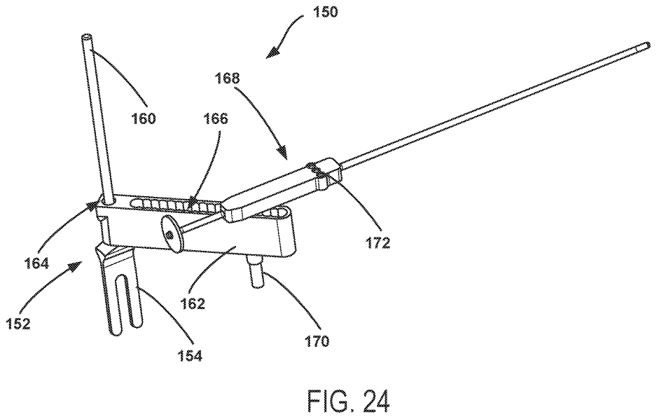

FIG. 23 illustrates a device 150 for initial pin alignment positioned adjacent an ankle anatomy. As shown in FIG. 23, the device 150 includes a gutter sword 152 including a flat portion 154 configured to be positioned between a tibia 156 and a talus 158 of a patient and an elongated rod 160 extending from the flat portion 154. The device 150 further includes a link 162 including a first through hole 164 configured to receive the elongated rod 160 of the gutter sword 152 and a plurality of second through holes 166 extending away from the first through hole 164 at varying distances. The device 150 further includes a pin alignment guide 168 including a rod 170 configured to be positioned in one of the plurality of second through holes 166 of the link 162. The pin alignment guide 168 includes one or more through holes 172 configured to receive one or more pins therethrough. FIG. 24 illustrates another variation of the device 150 for initial pin alignment.

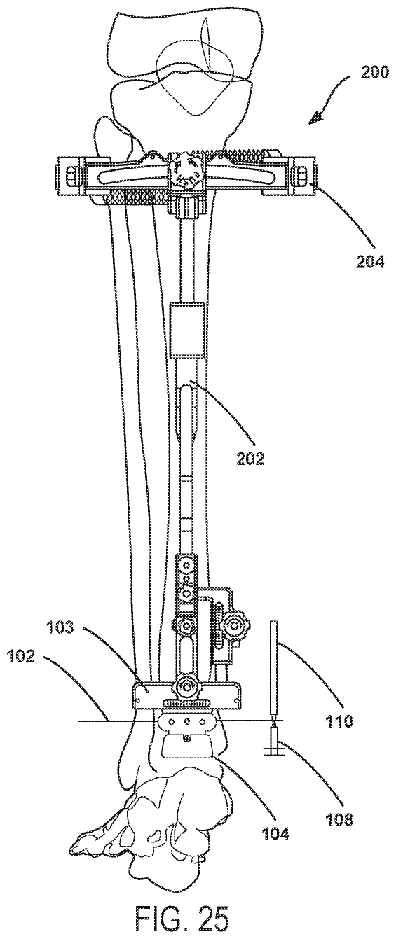

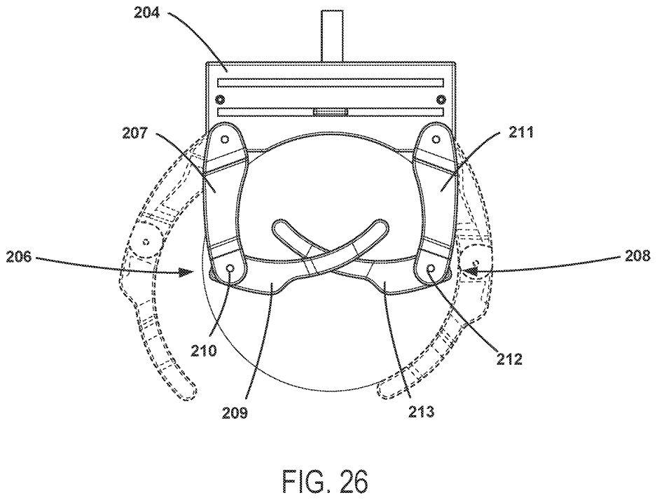

FIG. 25 illustrates an alignment system 200 including an external adjustment guide 103 and a reference wing 102 positioned adjacent an ankle anatomy. As shown in FIG. 25, the alignment system 200 further includes a rod 202 extending from the external adjustment guide 103 to a knee clamp 204. As shown in FIG. 26, the knee clamp 204 may include a first arm 206 and a second arm 208 that can open to varying diameters based on the anatomy of the patient. As further shown in FIG. 26, the first arm 206 may include a first portion 207 and a second portion 209 rotatably coupled to one another via a first pin joint 210, and the second arm 208 may include a first portion 211 and a second portion 213 rotatably coupled to one another via a second pin joint 212.

A kit for resecting a tibial bone is also disclosed. A kit includes a device 100 and a device 150 as described herein.

Methods disclosed herein can be used with any of the embodiments of the device 100, the device 150, and the kit as described herein.

A method of preparing a tibia for an implant includes positioning a pin in the tibia of a patient, positioning an external adjustment guide over the pin, removably coupling a reference wing 102 to the external adjustment guide 103, removably coupling a resection profile 104 to the reference wing 102, adjusting, via the external adjustment guide 103, a position of the resection profile 104 with respect to the tibia, and resecting a portion of the tibia using the resection profile 104 as a guide.

It should be understood that arrangements described herein are for purposes of example only. As such, those skilled in the art will appreciate that other arrangements and other elements (e.g. machines, interfaces, functions, orders, and groupings of functions, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and examples have been disclosed herein, other aspects and examples will be apparent to those skilled in the art. The various aspects and examples disclosed herein are for purposes of illustration and are not intended to be limiting, with the true scope being indicated by the following claims, along with the full scope of equivalents to which such claims are entitled. It is also to be understood that the terminology used herein is for the purpose of describing particular examples only, and is not intended to be limiting.

Example methods and systems are described herein. It should be understood that the words “example,” “exemplary,” and “illustrative” are used herein to mean “serving as an example, instance, or illustration.” Any example or feature described herein as being an “example,” being “exemplary,” or being “illustrative” is not necessarily to be construed as preferred or advantageous over other examples or features. The examples described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other examples may include more or less of each element shown in a given Figure. Further, some of the illustrated elements may be combined or omitted. Yet further, an example may include elements that are not illustrated in the Figures.

In the following description, numerous specific details are set forth to provide a thorough understanding of the disclosed concepts, which may be practiced without some or all of these particulars. In other instances, details of known devices and/or processes have been omitted to avoid unnecessarily obscuring the disclosure. While some concepts will be described in conjunction with specific examples, it will be understood that these examples are not intended to be limiting.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The limitations of the following claims are not written in means-plus-function format and are not intended to be interpreted based on 35 U.S.C. § 112(f), unless and until such claim limitations expressly use the phrase “means for” followed by a statement of function void of further structure.

By the term “about,” “approximately,” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. For example, in one embodiment, the term “about” can refer to ±5% of a given value.

Illustrative, non-exhaustive examples, which may or may not be claimed, of the subject matter according the present disclosure are provided below.

1. A device comprising:
a reference wing having a first end and a second end opposite the first end, wherein each of the first end and the second end of the reference wing includes a plurality of through holes;
a resection profile configured to be removably coupled to the reference wing between the first end and the second end; and
a lateral rod configured to be removably coupled to the reference wing via the plurality of through holes.

2. The device of claim 1, wherein the reference wing is semicurcular.

3. The device of claim 1, wherein the lateral rod comprises a biplane that is removably coupled to a lateral blade.

4. The device of claim 3, wherein the biplane comprises a first horizontal blade, a second horizontal blade, and a vertical blade, and wherein the first horizontal blade and the second horizontal blade indicate a superior talar resection and a native joint line, respectively.

5. The device of claim 3, wherein the biplane includes a shape feature that matches the plurality of through holes on the reference wing and further matches a mating hole of the lateral blade, wherein the shape feature prevents rotation of the biplane with respect to the reference wing when the shape feature is positioned through one of the plurality of through holes of the lateral blade.

6. The device of claim 5, wherein the mating hole of the lateral blade includes a canted coil spring to removably couple the lateral blade to the biplane.

7. The device of claim 1, wherein the reference wing includes a first cannulated rod and a second cannulated rod each positioned between the first end and the second end of the reference wing, and wherein the resection profile includes a first rod and a second rod configured to mate within the first cannulated rod and the second cannulated rod of the reference wing to thereby removably couple the resection profile to the reference wing.

8. The device of claim 7, wherein the first cannulated rod and the second cannulated rod each include a canted coil spring.

9. The device of claim 1, further comprising:
an external adjustment guide configured to be removably coupled to the reference wing between the first end and the second end, wherein the external adjustment guide is configured to be positioned on a first side of the reference wing, and wherein the resection profile is configured to be positioned on a second side of the reference wing.

10. The device of claim 9, wherein the reference wing includes a first cannulated rod and a second cannulated rod each positioned between the first end and the second end of the reference wing, and wherein the external adjustment guide includes a first hole and a second hole configured to receive the first cannulated rod and the second cannulated rod of the reference wing to thereby removably couple the external adjustment guide to the reference wing.

11. The device of claim 10, wherein the first hole and the second hole each include a canted coil spring.

12. The device of claim 1, wherein the resection profile comprises:
a plastic block; and
a metal insert configured to be positioned in the plastic block, wherein the metal insert is coupled to the plastic block via one or more fasteners.

13. The device of claim 12, wherein the metal insert includes one or more fluoroscopic alignment features.

14. The device of claim 1, wherein the reference wing includes a vertical alignment line, wherein the resection profile includes a horizontal alignment line, and wherein the vertical alignment line and the horizontal alignment line together form a crosshairs when the resection profile is removably coupled to the reference wing to thereby visualize a true anterior view of the device.

15. The device of claim 1, wherein the lateral rod comprises a lateral blade removably coupled to a block, wherein the block includes a plurality of k-wire holes corresponding to a joint line and a plurality of talar cuts corresponding to a plurality of sizes of implant.

16. A device comprising:
a gutter sword including a flat portion configured to be positioned between a tibia and a talus of a patient and an elongated rod extending from the flat portion;
a link including a first through hole configured to receive the elongated rod of the gutter sword and a plurality of second through holes extending away from the first through hole at varying distances; and
a pin alignment guide including a rod configured to be positioned in one of the plurality of second through holes of the link, wherein the pin alignment guide includes one or more through holes configured to receive one or more pins therethrough.

17. A kit comprising:
the device of claim 1; and
the device of claim 16.

18. A method of preparing a tibia for an implant, the method comprising:
positioning a pin in the tibia of a patient;
positioning an external adjustment guide over the pin;
removably coupling a reference wing to the external adjustment guide;
removably coupling a resection profile to the reference wing;
adjusting, via the external adjustment guide, a position of the resection profile with respect to the tibia; and
resecting a portion of the tibia using the resection profile as a guide.
